(19) United States
(12) Patent Application Publication
(10) Pub. No.: US 2009/0252767 A1
(43) Pub. Date: Oct. 8, 2009

(54) Water dispersible enteric coating formulation of nutraceutical and pharmaceutical dosage forms

(76) Inventors: Thomas Durig, Chadds Ford, PA (US); Derek Ronell Malone, Burlington, NJ (US); Yuda Zong, Wilmington, DE (US)

Correspondence Address: Hercules Incorporated;Hercules Plaza, 1313 North Market Street, Wilmington, DE, 19894-0001, US

(21) Appl. No.: 12/291,723

(22) Filed: Nov. 13, 2008

Related U.S. Application Data
(60) Provisional application No. 61/002,801, filed on Nov. 13, 2007.

(51) Int. Cl. A61K 9/00; A61K 47/38; A61K 47/04; A61K 47/10; C09D 193/02

(52) U.S. Cl. 424/400; 514/781; 514/779; 514/769; 514/772; 524/77

(57) ABSTRACT

The present invention relates to formulations for use as enteric coatings. More particularly, the present invention relates to a formulation comprising a dry blend of food grade ingredients that can be readily dispersed in water. This dispersion exhibits low viscosity and can easily be coated onto solid dosage forms through spraying and the like to provide an enteric coating on the solid dosage form.

### FIELD OF THE INVENTION

The present invention relates to formulations for use as enteric coatings. More particularly, the present invention relates to a formulation comprising a dry blend of food grade ingredients that can be readily dispersed in water and coated onto solid dosage forms to provide an enteric coating thereon.

### BACKGROUND OF THE INVENTION

Enteric film coatings are applied to oral dosage forms to delay the release of active ingredients until the dosage form has passed through the acidic environment of the stomach and has reached the near-neutral environment of the proximal small intestine. The physical chemical environment of the stomach and gastric physiology are highly variable, subject to multiple factors such as disease state, medication, age, and eating. For example in the fasted state stomach, the pH is less than 2 in healthy individuals, and gastric emptying occurs approximately every 30 minutes. However in the fed state (immediately after a meal), gastric emptying is delayed for 2 to 4 hours and gastric pH can be as high as pH 4.

It can therefore be seen that an ideal enteric coating system would have to be flexible. The majority of enterically coated dosage forms are recommended to be taken on an empty stomach. Such coatings would therefore have to be resistant to the acidic stomach environment for a relatively short time and would not be expected to be subjected to strong mechanical attrition in the stomach. On the other hand to allow for possible ingestion in the fed state, or where subsequent release from the intestine is not intended to be immediate, the coating will have to be sufficiently robust to withstand prolonged attrition in the stomach or to generally release more slowly in the alkaline environment.

There is a long history of use of enteric coatings on tablets and smaller multi-particulate dosage forms in the pharmaceutical industry. Generally polymers with acidic functional groups are chosen for enteric coatings. In the acid environment of the stomach these acid groups of the polymers are un-ionized, thus rendering the polymer water insoluble. However in the more neutral and alkaline pH of the intestine (pH 6.8-7.2), the functional groups ionize and the polymer film coating becomes water soluble.

Examples of enteric film coatings include methacrylic acid copolymers, polyvinyl acetate phthalate, cellulose acetate phtallate, hydroxypropyl methylcellulose phthalate and hydroxypropyl methylcellulose acetylsuccinate. Traditionally these water soluble coatings have been applied from organic solvent based coating solutions. However due to environmental and safety concerns and the costs associated with organic solvent coating, aqueous based dispersions and pseudo-latex systems of some of the above polymers are increasingly preferred. However, none of the above named polymers are approved for food use, including nutritional supplements, such as nutraceuticals. None of the above polymers are found in the Food Chemicals Codex (FCC) and none of the above polymers have direct food additive status or have generally regarded as safe (GRAS) status.

Several strategies have been developed to provide for food grade enteric coatings for nutraceuticals and other items classified as food.

An aqueous ethylcellulose (EC) based pseudo-latex has been used in conjunction with sodium alginate. This product is marketed as Nutrateric™ nutritional enteric coating system by Coloroon Inc. of Westpoint, Pa. This coating is supplied as a two component system in the form of an aqueous anmmoniated EC dispersion with 25% solids and a separate container of sodium alginate in powder form. To prepare the final coating solution, the sodium alginate is fist dispersed and dissolved in water for 60 minutes and EC dispersion is then added to the alginate solution, ensuring that the amount of water used is appropriate to achieve a final recommended dispersed solids concentration of 10% by weight. This relatively low solids concentration is recommended to ensure a sufficiently uniform coating. This relatively low solids concentration is recommended because the viscosity of this solution is inherently high. At 10% solids concentration, the coatings system has a viscosity of 430 cps at 22° C., when measured with a Brookfield Model LVT viscometer using spindle #1 at 100 rpm. For typical pumping and spraying equipment used in aqueous film coating, this is a very high viscosity and higher solids would typically be difficult to process. Such high viscosities (above 200 cps) also have a significant effect on droplet size and spreadability of the coating, thus negatively impacting film uniformity. The low solids concentration (10% by weight) is especially problematic for large scale coating of soft gelatin capsules, where prolonged exposure to high amounts of water and heat may lead to deleterious effect such as softening of the gelatin capsule walls. Furthermore, the lack of spreadability of the coating due to its relatively high viscosity can lead to blistering and non uniformity effects.

An alternative approach is the use of shellac on its own or in combination with other additives.

Shellac is a natural, food approved, resinous material obtained from the exudate of the insect Karria lacca. It is a complex mixture of materials. The two main components with enteric properties being shelloic and aleuritic acid. While shellac is well known as a material with enteric-like properties, it has a number of drawbacks. Due to insolubility in water, shellac has traditionally been used in the form of organic solvent based solutions. Additionally in its natural state, shellac is generally not soluble below a pH of 7.5 to 8.0. Rather shellac films simply soften and disintegrate after immersion in water for a number of hours. This is problematic as enteric coatings should generally be soluble or rupturable at approximately pH 6.8. Lastly shellac coatings have been reported to undergo esterification during aging, rendering the film completely water insoluble even in alkaline pH.

To obviate the use of solvents, neutralized aqueous shellac solutions are commercially available. EP 1 579 771 A1 describes a water based shellac dispersion which comprises shellac, a basic amino acid, a basic phosphate and water. The basic amino acid being selected from the group consisting of arginine, lysine and ornithine.

Several forms of aqueous ammoniated shellac dispersions are also commercially available, for example Certiseal® FC 300A film coat product, manufactured by Mantrose Haeuser, a subsidiary of RPM Corporation. Esterificaton of the shellac is also limited in these systems as shellac forms a salt with the ammonia or protonated amino acid.

However these systems do not address directly the need for an enteric food grade coating which is soluble or rupturable at a pH of 6.8.

In US Patent 2007/0071821A, an enteric coating formulation in the form of a spray solution or suspension is disclosed. This system comprises shellac in aqueous salt form and sodium alginate, preferably in equal concentrations. An aqueous solution of an alkali salt of shellac is prepared by first dissolving the shellac in 55° C. hot water, then adding 10% ammonium hydrogen carbonate and heating to 60° C. and stirring for 30 minutes. Separately, a sodium alginate solution is prepared and the two solutions are then blended together. The system, when coated onto a dosage form, is claimed to be acid resistant yet rapidly disintegrates in pH 6.8 buffer. However, the blend of shellac and sodium alginate as described in US Patent 2007/0071821A would generally have a viscosity exceeding 400 cps at a 20% solids concentration. In order to accommodate these relatively high viscosities, relatively dilute coating solutions (6-10% solids) would therefore have to be used to facilitate spraying and pumping in commercially available coating equipment.

The above approaches describe enteric coatings composed of food approved ingredients, which are either pH sensitive or more time dependant in terms of their delayed release mechanism. However, all these systems require multiple, time consuming preparation steps, often requiring two separate solutions to be made with additional dilution requirements and potential for error. Alternately, the systems require the use of pre-made dispersions of EC or shellac, which then require further solution steps and blending adding cost and/or time to the manufacturing process.

In the case of pre-made dispersions, further cost is incurred due to the need to store and ship the added bulk of water. Additionally, these pre-made aqueous systems require precautions in terms of microbial contamination and physical and chemical instability. Moreover enteric coatings are generally applied in relatively high amounts. A five to ten percent weight gain during a coating step is typical. This amount of weight gain requires relatively long coating runs of 2 to 4 hours at typical, industry standard application rates. As a point of reference, it is typical to apply aesthetic, non-functional coatings at 3% weight gain in approximately an hour.

In summary, a need exists for a pH sensitive, food grade enteric coating formulation in a dry form that can be readily dispersed in water, as little as 1 hour before use, in a single, simple preparation step. Moreover, the ability to spray such a system at relatively high solids (15-20%) and to readily adjust for strength without the need for more coating weight is advantageous, allowing for more efficient coating operations. Also, a need exists for a dry form of a shellac that can be readily dispersed in water to produce coatings comprising shellac on various substrates.

### BRIEF DESCRIPTION OF THE INVENTION

The present invention relates to a dry powder formulation useful for producing a sprayable dispersion enteric coating. The dry powder formulation comprising a food grade shellac, an ammonium salt, such as ammonium carbonate or ammonium bicarbonate, and an anionic polymer. The dry powder formulation when dispersed in water is capable of producing a sprayable dispersion enteric coating. This coating at 15% solids in water has a viscosity of below 100 cps at about 22° C. when measured with a Brookfield LTV viscometer with a #1 spindle at 100 rpm.

A formulation for a dry blend of food grade ingredients that can be readily dispersed in water and the dispersion coated onto solid dosage forms to provide an enteric coating is disclosed. When dispersed in hot water, the mixture is ready for coating onto solid dosage forms, such as tablets, capsules and small particulates after about 60 minutes of dispersing the dry blend into water. The resultant coating is pH sensitive. When subjected to a disintegration test in acidic simulated gastric fluid, the dosage forms coated with the inventive water dispersible powder blend resist break-up for about 60 minutes, but disintegrate within about 90 minutes after subsequent immersion in neutral (pH 6.8) simulated intestinal fluid. The water dispersible powder blend comprises shellac, ammonium carbonate, and an anionic polymer such as sodium carboxymethyl cellulose (CMC), sodium alginate or pectin. Optionally, the water dispersible powder blend further comprises one or more plasticizers chosen from the group consisting of glycerine, mineral oil, triacetin, polyethylene glycol, glyceryl monostearate, mono-acetylated triglycerides and polysorbate. Optionally, the water dispersible powder blend may comprise pigments, and detackifiers such as titanium dioxide, talc, iron oxide, and natural colors. Due to the unexpected ability to accommodate pigment loads exceeding 40% while maintaining pH sensitivity, opaque coatings on solid dosage forms with high hiding power and good “handfeel” are possible. If no pigments are included in the water dispersible powder blend of the present invention, the resultant coating is clear, translucent with a golden hue which is especially useful for coating soft gel capsules, in particular oil containing soft gel capsules such as fish oil. In this case, the enteric coating produced from the water dispersible powder blend helps prevent the premature release of fish oil in the stomach, thus reducing the chance of reflux and fish odor and after taste. When the water dispersible powder blend formulations of the present invention are dispersed in about 50 to 70° C. hot water at 20% solids concentration, they are characterized by viscosities of less than 200 cps.

The present invention also relates to a dry form of shellac, anionic polymer and ammonium carbonate which can be readily dispersed in water to produce shellac coatings on various substrates.

The present invention also relates to a process for producing the sprayable dispersion enteric coating comprising the steps of dry blending a food grade shellac, ammonium carbonate, an anionic polymer, one or more plasticizers chosen from glycerine, mineral oil, triacetin, polyethylene glycol, glyceryl monostearate and polysorbate, and, optionally, and, optionally, pigments, and detackifiers such as titanium dioxide, talc, iron oxides and natural colors together to form a dry powder formulation. The dry powder formulation is then dispersed in about 50 to 70° C. hot water. The dispersion is stirred for a sufficient period of time to produce a low viscosity sprayable dispersion wherein the low viscosity sprayable dispersion at 15% solids in water has a viscosity of below 100 cps at about 22° C. when measured with a Brookfield LTV viscometer with a #1 spindle at 100 rpm.

The present invention also relates to a process for producing a solid dosage form having an enteric coating and the resultant enteric coated nutraceutical or pharmaceutical wherein the above described the sprayable dispersion enteric coating is sprayed as a low viscosity sprayable dispersion onto a nutraceutical or pharmaceutical active ingredient in a solid dosage form to produce an enteric coating on the nutraceutical or pharmaceutical active ingredient in a solid dosage form.

### DETAILED DESCRIPTION OF THE INVENTION

It has been found that food grade shellac can be dry blended with other food grade ingredients to form a water dispersible powder blend which is readily dispersible and useful in producing enteric coating, suitable for coating on to nutraceutical and pharmaceutical solid dosage forms, such as tablets, capsules and small particulates. In addition to shellac, the water dispersible powder blend comprises ammonium carbonate, and an anionic polymer such as sodium carboxymethyl cellulose (CMC), sodium alginate or pectin. Optionally, the water dispersible powder blend comprises one or more plasticizers chosen from glycerine, mineral oil, triacetin, polyethylene glycol, glyceryl monostearate and polysorbate. Optionally, the water dispersible powder blend further comprises pigments, and detackifiers such as titanium dioxide, talc, iron oxide. Additional components such as natural colors, various carbohydrate derivatives such as hypromellose, hydroxypropyl cellulose, carboxymethyl starch, carageenan and xanthan may also be used in the water dispersible powder blend of the present invention.

While not excluding other grades of shellac, a preferred type is Orange Dewaxed Shellac compliant with the monographs of the USP and FCC. For optimal blending and water dispersion, the shellac flakes are milled prior to blending with the other ingredients of the water dispersible powder blend and resultant coating. Suitable milling and size reduction can be achieved with an impact mill for example a Fitzpatrick type hammermill. Particle size distributions where 99% of the particles by volume are smaller than 1000 microns are preferred. The amount of shellac of use in the water dispersible powder blend of the present invention is in the range of from about 20% to about 75% by weight of the blend and coating, more preferably from about 30% to about 70% by weight of the blend and coating.

The preferred anionic polymer for use in the water dispersible powder blend comprises sodium carboxymethyl cellulose (CMC). The preferred CMC being a low viscosity grade such as Aqualon® CMC 7L2P, marketed by Aqualon, a Business Unit of Hercules Incorporated. Various grades of sodium alginate have also been found suitable for the anionic polymer for use in the water dispersible powder blend of the invention. The amount of anionic polymer of use in the water dispersible powder blend and resultant enteric coating of the present invention is in the range of from about 1% to about 18% by weight of the blend and coating, more preferably from about 2% to about 12% by weight of the blend and coating.

The water dispersible powder blend and resultant enteric coating also comprises an amount of ammonium carbonate. The amount of an ammonium salt, such as ammonium carbonate or ammonium bicarbonate, of use in the water dispersible powder blend and resultant enteric coating of the present invention is in the range of from about 1.5% to about 9% by weight of the blend and coating, more preferably from about 1.5% to about 8% by weight of the blend and coating.

If the water dispersible powder blend also optionally comprises a plasticizer selected from the group consisting of glycerine, mineral oil, triacetin, polyethylene glycol, mono-acetylated triglycerides, glyceryl monostearate and polysorbate. If glycerine is the plasticizer, then it may be used in an amount in the range of from about 3% to about 10% by weight of the blend, more preferably from about 5% to about 10% by weight of the blend. If mineral oil is the plasticizer, then it may be used in an amount in the range of from about 3% to about 9%, more preferably from about 5% to about 7% by weight of the blend. If glyceryl monostearate is the plasticizer, then it may be used in an amount in the range of from about 3% to about 12%, more preferably from about 5% to about 10% by weight. If polysorbate 80 is the plasticizer, then it may be used in an amount in the range of from about 3% to about 12%, more preferably from about 5% to about 10% by weight. If mono-acetylated triglyceride is the plasticizer, then it may be used in an amount in the range of from about 3% to about 12%, more preferably from about 5% to about 10% by weight.

A unique and surprising feature of the food grade enteric system in a dry form is that levels of up to about 70% by weight of inorganic pigments, such as talc or titanium dioxide (TiO₂), can be accommodated while maintaining pH sensitivity and mechanical strength and smooth surface texture of enteric coatings produced from the food grade enteric system in a dry form. In typical pharmaceutical film coatings, the upper level for pigments such as these is usually 40% by weight, with poor mechanical properties and chalkiness resulting at these higher levels. Surprisingly, in spite of the high pigment levels, and in spite of high solids levels of 20% by weight, a dispersion of the water dispersible powder blend of the present invention in water has remarkably low viscosities of less than 100 cps, but frequently lower than 30 cps when measured using a Brookfield LTV viscometer at about room temperature (22° C.), using a #1 spindle at 100 rpm. The resulting enteric coating fluid is therefore sprayable at very high rates, resulting in first processing, excellent spreadability, film uniformity and smoothness. Processing time can further be enhanced by reconstituting the enteric coating fluid at 25% by weight solids while still maintaining viscosity below 300 cps. Low viscosity is also a surprising feature of the unpigmented, clear coatings which at 15-20% solids typically vary from 20-100 cps.

Other food grade enteric systems such as the aqueous EC pseudo latex system referred to earlier have much higher viscosities (430 cps at 10% solids by weight). Other functional enteric coating systems such as methacrylic acid co-polymer pseudo latex systems are available as low viscosity dispersions. However, none of these low viscosity enteric dispersions can be readily formed by dispersing a powder composition in water for 60 minutes prior to use using simple stirring equipment, while simultaneously meeting the requirements of a nutraceutical coating system, whose ingredients are approved as direct food additives and can be found in the FCC, the FDA direct food additive list or the FDA GRAS list. The low viscosity of the dispersions of the food grade enteric system of the present invention results in excellent droplet spread ability on the dosage form substrate, resulting in smooth coatings but also high adhesion due to the ability to fill into surface imperfections and capillary pores.

Typical compositional ranges for these pigmented systems are as follows:

Shellac 75-20% by weight, ammonium carbonate 9-1.5% by weight, CMC 18-1% by weight, if sodium alginate is included 7-1% by weight, if glycerine is included 10-3% by weight, if mineral oil is included 9-3% by weight, if glyceryl monostearate is included 12-3% by weight, if polysorbate 80 is included 12-3% by weight, if talc is included 60-2% by weight, if titanium dioxide is included 60-2% by weight. A more preferred range is: Shellac 70%-30% by weight, ammonium carbonate 8% -1.5% by weight, CMC 12-2% by weight, if sodium alginate is included 6-2% by weight, if glycerine is included 10-5% by weight, if mineral oil is included 7-5% by weight, if glyceryl monostearate is included 10-5% by weight, if polysorbate 80 is included 10-5% by weight, if talc is included 24-2% by weight and if TiO₂ is included 24-2% by weight.

Among the plasticizers of use in the present invention, glycerine is the most preferred due to its universal status as a food plasticizer. Furthermore, other plasticizers like triacetin, while of utility in the present invention, have surprisingly showed a potential to sometimes cause discoloration on aging. This is not seen with glycerine. For coatings that are to be applied to soft gel capsules, combinations of plasticizers are most preferred, for instance, the combination of glycerine with mineral oil or the combination of polysorbate 80 with glyceryl monostearate.

If no pigment is included in the food grade enteric system of the present invention, the resultant enteric coatings are translucent, slightly gold colored, clear coating systems which are especially useful for coating soft gel capsules.

Various effective combinations, highlighting the versatility of the system are discussed in the examples below.

The food grade enteric system in a dry form of the present invention can be manufactured by any suitable powder blending technique. Smaller lots can be readily prepared in a Cuisinart type food processor or a Hobart type planetary mixer. Larger quantities can also be manufactured in high and medium shear blenders such as for example, a Colette-Gral mixer, ribbon blenders and V-blenders. No blender specific issues have been identified, thus the food grade enteric system in a dry form of the present invention is expected to be able to be manufactured in a host of other blending equipment.

Typical preparation would involve any suitable powder blending technique for blending the shellac, anionic polymers, pigments, such as talc or titanium dioxide for example, for about 5 to 10 minutes, followed by addition of plasticizer over a period of about 3 to 5 minutes, after this blending may be continued for about another 3 minutes. The resulting blend is dry to the touch and can be stored in suitable containers, such as plastic lined fiber drums or boxes, until use.

When the water dispersible powder blend is dispersed in hot water, about 55° C. to 70° C., while stirring, the resulting dispersion is ready for coating pharmaceutical solid dosage forms, such as tablets, capsules and small particulates, after 60 minutes of stirring. The resultant enteric coating is pH sensitive. When subjected to a standard USP Disintegration Test in acidic simulated gastric fluid without discs, tablets coated with the enteric coating of the present invention resist break-up for 60 minutes, but disintegrate within 90 minutes after subsequent disintegration testing in neutral (pH 6.8) simulated intestinal fluid with discs. When soft gelatin capsules coated with the enteric coating of the present invention are subjected to a standard USP Disintegration Test in acidic simulated gastric fluid without discs, the capsules will resist break up for 60 minutes, but will rupture within 60 minutes after subsequent disintegration testing in simulated intestinal fluid (pH 6.8) without discs. However as illustrated below, the enteric coating of the present invention can also be formulated to meet a number of different performance targets. For example, it can be formulated to be more robust, thus allowing for acid resistance for 1 hour in a USP Disintegration Test with discs, followed by disintegration in pH 6.8 buffer within 60 minutes or 90 minutes. As indicated earlier, if enteric dosage forms are ingested in the fed state, they will reside in the stomach for a period of hours. The tablets may therefore be exposed to mechanical attrition in an acidic environment for extended periods. This can be simulated by including discs in the disintegration apparatus, which collide with the tablet during each oscillation of the disintegration apparatus.

Viscosities of the dispersions were determined using a Brookfield LTV viscometer with a #1 spindle and at 100 rpm, unless noted otherwise.

The examples are presented to illustrate the invention, parts and percentages being by weight, unless otherwise indicated.

### EXAMPLES

### Example 1 (Comparative)

A coating formulation in the form of a sprayable aqueous dispersion was produced by weighing out the correct amounts of polymers and ingredients and then pre-dissolving shellac and ammonium carbonate in 60° C. hot water for 60 minutes while stirring. A separate aqueous solution of CMC (Aqualon® CMC 7 L2P) was prepared by dispersing CMC into cold water while stirring for 60 minutes until dissolved. These two solutions were then blended together and talc, titanium and triacetin were added to the final coating composition. The final coating dispersion was stirred for 30 minutes to ensure homogeneity

The solids composition by weight without water is given below:

| | |
|---|---|
| Orange Dewaxed Shellac | 34.5 |
| Ammonium carbonate | 2.5 |
| CMC 7L2P | 3 |
| Titanium dioxide | 41 |
| Talc | 14 |
| Triacetin | 5 |

The viscosity of this final coating composition at 20% solids was measured as 20 cps using a Brookfield viscometer with spindle No. 1 at 100 rpm. This viscosity retained similar for 72 hours. When the final coating composition was prepared as a 25% by weight solids composition, the viscosity of the final coating composition was 75 cps which is still remarkably low for a typical film coating solution where the polymer is fully dissolved in the solvent. This allows for high spray rates and fast processing using typical coating pans and spray guns used in the nutraceutical and pharmaceutical industry.

When the final coating composition was applied onto round, concave multi-vitamin tablets (˜200mg initial tablet weight) to a 4% weight gain in a Vector HS coater with 1 kg tablet capacity, the tablets were resistant to disintegration testing in 0.1N HCl (pH 1.2) solution for one hour with discs, and when subsequently disintegration tested in pH 6.8 phosphate buffer with discs, the tablets disintegrated in less than 90 minutes.

### Example 2

The same solids composition as shown in example 1 (comparative) was dry blended in a food processor, by blending the shellac, ammonium carbonate, CMC, talc and titanium for 3 minutes. The triacetin was then added gradually over a period of 2 minutes while continuing to blend. The final dry blend was then mixed a further 3 minutes.

The dry powder formulation was then dispersed in 60° C. hot water while stirring. A uniform sprayable dispersion resulted within 60 minutes. The 20% solids dispersion had a viscosity of 20 cps when tested using a Brookfield LTV viscometer using a #1 spindle at 100 rpm. Similar viscosity was maintained for 72 hours. The coating solution was also sprayable at 25% solids where the viscosity was 75 cps.

When coated on the same lot of multivitamins to a 4% weight gain, the tablets coated with the dry coating dispersion of example 2 were resistant to disintegration in pH 1.2 for 1 hour (with discs) and fully disintegrated in less than 90 minutes when subsequently subjected to disintegration in pH 6.8 phosphate buffer with discs.

This illustrates the advantage of the dry powder formulation as an enteric coating delivery system which can be pared in advance and stored for al extended period of time and transported without concern for stability and without the added cost and bulk of shipping water, but can be prepared in a single step for coating in as little as 60 minutes, with good results.

### Example 3

To adjust the coating so that it resists disintegration in acid media for 1 hour without the use of discs in the disintegration apparatus, but results in rapid disintegration in pH 6.8 phosphate buffer, the following dry powder formulation was prepared as described for powder blending in Example 2:

| | |
|---|---|
| Orange Dewaxed Shellac | 23% |
| CMC 7L2P | 3% |
| Ammonium Carbonate | 2.5% |
| Titanium Dioxide | 28% |
| Talc | 43.5% |

It can be seen that this composition has an inorganic pigment load of 71.5% by weight. The dry powder formulation was dispersed in 55° C. hot water for 1 hour while stirring. The viscosity of 20% solids dispersion was 19-25 cps and remained similar for 72 hours. The sprayable dispersion was sprayed onto multi-vitamin tablets from the same tablet lot as described in Example 1 (comparative). The same coating equipment was also used.

The tablets were coated to 4, 5, 6 and 7% weight gain. The tablets with 7% weight gain were found to meet the dual, sequential requirements of resistance to disintegration in pH 1.2 (0.1N HCl) for 1 hour without discs, followed by disintegration within 1 hour at pH 6.8 (phosphate buffer, with discs). All other coating levels failed to meet the sequential requirements.

### Example 4 (Comparative)

The same composition as shown in Example 3 but without the presence of CMC was prepared as follows:

| | |
|---|---|
| Orange Dewaxed Shellac | 23% |
| Ammonium carbonate | 2.5% |
| Titanium dioxide | 31% |
| Talc | 43.5% |

The dry powder formulation was prepared as previously described in Example 2. A 20% solids dispersion was made by adding the blend to 55° C. hot water while stirring for 60 minutes. A viscosity of 19 cps was measured for the 20% solids dispersion.

Using the same lot of tablets described in Example 1 (Comparative) and the same coating equipment, the tablets were coated to 4, 5, 6 and 7% weight gain. None of the coating trials were found to meet the dual, sequential requirements of resistance to disintegration in pH 1.2 (0.1N HCl) for 1 hour, followed by disintegration within 1 hour at pH 6.8 (phosphate buffer). All coating levels were acid resistant, but even at the lowest level of 4% weight gain the coating did not filly dissolve in pH 6.8 phosphate buffer during disintegration. Using lower coating levels resulted in highly variable results with some tablets passing and some failing due to non-uniform coating. Example 4 (Comparative) therefore demonstrates the need to include an anionic polymer, such as CMC, in order to produce a dry, water dispersible coating system that can sequentially withstand disintegration in pH1.2 for 60 minutes, and then completely disintegrate within another 90 minutes when changed over to pH 6.8 and subjected to further disintegration.

### Example 5 (Comparative)

To further improve the disintegration performance of the coating system, the following variation on Example 3 was prepared:

| | |
|---|---|
| Orange Dewaxed Shellac | 23% |
| CMC 7L2P | 2% |
| Ammonium Carbonate | 2.5% |
| Sodium Alginate | 3% |
| Titanium dioxide | 28% |
| Talc | 41.5% |

The dry powder formulation was prepared as previously described in Example 2. A 20% solids dispersion was made by adding the blend to 55° C. hot water while stirring for 60 minutes. A viscosity of 144 cps was measured for the 20% solids dispersion.

Using the same lot of tablets described in Example 1 (Comparative) and the same coating equipment, the tablets were coated to 4, 5, 6 and 7% weight gain.

It was found that the coating trials with 5, 6 and 7% weight gain all passed the two stage disintegration test, with resistance to disintegration for 60 minutes in pH 1.2 (0.1N HCl) without discs, followed by complete disintegration in pH 6.8 phosphate buffer (with discs) upon testing for another 90 minutes.

Further examples of the enteric coating system are given below

### Example 6

A dry powder formulation was prepared as follows:

| | |
|---|---|
| Orange Dewaxed Shellac | 23% |
| Ammonium carbonate | 3% |
| CMC 7L2P | 2% |
| Sodium alginate | 3% |
| Titanium dioxide | 28% |
| Talc | 37% |
| Glycerine | 4% |

A 20% solids blend was dispersed in 55° C. hot water while stirring for 1 hour. The coating dispersion was sprayed onto caplet shaped garlic tablets (initial tablet weight ˜1 gram) in a Vector HS coating pan with 1 kg capacity. The coated garlic tablets were then subjected to disintegration testing in pH 1.2 (0.1N HCl) solution without discs for 60 minutes, followed by disintegration testing with discs in pH 6.8 phosphate buffer. It was found that when coated to a 7% weight gain, the tablets resisted disintegration in pH 1.2 media for 60 minutes but disintegrated during the subsequent phosphate buffer stage (pH 6.8) in less than 90 minutes.

### Example 7

As for Example 6 but triacetin was substituted for glycerin. The outcome was similar.

### Example 8

A dry powder formulation was prepared as previously described in Example 2 with the following components:

| | |
|---|---|
| Orange Dewaxed Shellac | 35% |
| Ammonium carbonate | 3.5% |
| CMC 7L2P | 2% |
| Sodium alginate | 6% |
| Titanium dioxide | 22% |
| Talc | 22.5% |
| Glycerine | 9%. |

A 20% solids blend was dispersed in 65° C. hot water while stirring for 1 hour. The coating dispersion was sprayed onto 2 kg of caplet shaped garlic tablets (initial tablet weight˜1 gram) in a 15 inch Ohara Labcoat IIX coating pan. The coated garlic tablets were then subjected to disintegration testing in pH 1.2 (0.1N HCl) solution without discs for an hour, followed by disintegration testing with discs in pH 6.8 phosphate buffer. It was found that when coated to a 4% weight gain, the tablets resisted disintegration in pH 1.2 media for one hour but disintegrated during the subsequent phosphate buffer stage (pH 6.8) in less than 90 minutes.

### Example 9

A dry powder formulation was prepared as previously described in Example 2 with the following components:

| | |
|---|---|
| Orange Dewaxed Shellac | 35% |
| Ammonium carbonate | 3% |
| CMC 7L2P | 2% |
| Sodium alginate | 3% |
| Titanium dioxide | 24% |
| Talc | 20% |
| Glycerine | 8% |
| Mineral Oil | 5% |

A 15% solids blend was dispersed in 65° C. hot water while stirring for 1 hour. The viscosity of this dispersion was 17.5 cps measured with a Brookfield LVT viscometer using spindle #1 at 100 rpm. The coating dispersion was sprayed onto 2 kg of fish oil containing soft gel capsules (initial capsule weight˜1.7 gram) in a 15 inch Ohara Labcoat IIX coating pan. The soft gel capsules were then subjected to disintegration testing in pH 1.2 (0.1N HCl) solution without discs for an hour, followed by disintegration testing in pH 6.8 phosphate buffer without discs. It was found that when coated to a 4% weight gain, the capsules resisted disintegration in pH 1.5 media for one hour but ruptured and leaked oil within 30 minutes during the subsequent phosphate buffer stage (pH 6.8). The results for capsules coated at 5% and 6% weight gain were substantially similar.

### Example 10

A dry powder formulation was prepared as previously described in Example 2 with the following components:

| | |
|---|---|
| Orange Dewaxed Shellac | 65% |
| Ammonium carbonate | 5% |
| CMC 7L2P | 11% |
| Talc | 4% |
| Glycerine | 8% |
| Mineral Oil | 7% |

A 15% solids blend was dispersed in 65° C. hot water while stirring for 1 hour. The viscosity of this dispersion was below 15 cps measured with a Brookfield LVT viscometer using spindle #1 at 100 rpm. The coating dispersion was sprayed onto 2 kg of fish oil containing soft gel capsules (initial capsule weight˜1.7 gram) in a 15 inch Ohara Labcoat IIX coating pan. The soft gel capsules were then subjected to disintegration testing in pH 1.2 (0.1N HCl) solution without discs for an hour, followed by disintegration testing in pH 6.8 phosphate buffer without discs. It was found that when coated to a 4% weight gain, the capsules resisted disintegration in pH 1.2 media for one hour but ruptured and leaked oil within 30 minutes during the subsequent phosphate buffer stage (pH 6.8).

### Example 11

A dry powder formulation was prepared as previously described in Example 2 with the following components:

| | |
|---|---|
| Orange Dewaxed Shellac | 70% |
| Ammonium carbonate | 7.5% |
| CMC 7L2P | 5.5% |
| Polysorbate 80 | 8.5% |
| Glyceryl Monostearate | 8.5% |

A 15% solids blend was dispersed in 65° C. hot water while stirring for 1 hour. The viscosity of this dispersion was 20.4 cps measured with a Brookfield LVT viscometer using spindle #1 at 100 rpm. At 20% solids, the viscosity was 75 cps. The 15% coating dispersion was sprayed onto 2 kg of fish oil containing soft gel capsules (initial capsule weight˜1.7 gram) in a 15 inch Ohara Labcoat IIX coating pan. The soft gel capsules were then subjected to disintegration testing in pH 1.2 (0.1N HCl) solution without discs for an hour, followed by disintegration testing in pH 6.8 phosphate buffer without discs. It was found that when coated to a 5% weight gain, the capsules resisted disintegration in pH 1.2 media for one hour but ruptured and leaked oil within 40 minutes during the subsequent phosphate buffer stage (pH 6.8).

### Example 12

A dry powder formulation was prepared as previously described in Example 2 with the following components:

| | | |
|---|---|---|
| Orange Dewaxed Shellac | 66 | parts by weight |
| Ammonium carbonate | 7 | parts by weight |
| CMC 7L2P | 11 | parts by weight |
| Talc | 10 | parts by weight |
| Mineral oil | 7 | parts by weight |
| Glycerine | 9 | parts by weight |

A 15% solids blend was dispersed in 65° C. hot water while stirring for 1 hour. The viscosity of this dispersion was 19 cps measured with a Brookfield LVT viscometer using spindle #1 at 100 rpm. The 15% coating dispersion was sprayed onto 2 kg of S-adenosyl methionine (SAM-e) tablets in a 15 inch Ohara Labcoat IIX coating pan. The tablets were then subjected to disintegration testing in pH 1.2 (0.1N HCl) solution without discs for an hour, followed by disintegration testing in pH 6.8 phosphate buffer with discs. It was found that when coated to a 2% weight gain, the tablets resisted disintegration in pH 1.5 media for one hour but disintegrated within 90 minutes during the subsequent phosphate buffer stage (pH 6.8).

The examples are merely set forth for illustrative purposes all parts and percentages being by weight, unless otherwise indicated. It is to be understood that other modifications of the present invention can be made by skilled artisans in the related industry without departing from the spirit and scope of the invention.

What is claimed is:

1. A dry powder formulation useful for producing a sprayable dispersion enteric coating, comprising:
a food grade shellac,
an ammonium salt, and
an anionic polymer
wherein the sprayable dispersion enteric coating at 15% solids in water has a viscosity of below 100 cps at about 22° C. when measured with a Brookfield LVT viscometer with a #1 spindle at 100 rpm.

2. The dry powder formulation of claim 1 wherein the anionic polymer is selected from the group consisting of sodium carboxymethyl cellulose (CMC), sodium alginate and pectin.

3. The dry powder formulation of claim 2 wherein the anionic polymer comprises sodium carboxymethyl cellulose (CMC) in an amount in the range of from about 1% to about 18% by weight of the dry powder formulation.

4. The dry powder formulation of claim 3 wherein the anionic polymer further comprises sodium alginate in an amount in the range of from about 1% to about 7% by weight of the dry powder formulation.

5. The dry powder formulation of claim 1 wherein the food grade shellac is Orange Dewaxed Shellac in an amount in the range of from about 20% to about 75% by weight of the dry powder formulation.

6. The dry powder formulation of claim 1 wherein the ammonium salt of use in the dry powder formulation comprises ammonium carbonate in an amount in the range of from about 1.5% to about 9% by weight of the dry powder formulation.

7. The dry powder formulation of claim 1 further comprising one or more plasticizers chosen from the group consisting of glycerine, mineral oil, triacetin, polyethylene glycol, glyceryl monostearate, monoacetylated triglyceride and polysorbate.

8. The dry powder formulation of claim 1 further comprising an inorganic pigment in an amount up to about 70% by weight of the dry powder formulation.

9. The dry powder formulation of claim 8 wherein the inorganic pigment is selected from the group consisting of titanium dioxide, talc and iron oxides.

10. The dry powder formulation of claim 6 wherein the dry powder formulation comprises food grade shellac in the range of from about 30% to about 70 by weight, ammonium carbonate in the range of from about 1.5% to about 8% by weight, CMC in the range of from about 2% to about 12% by weight, glycerine in the range of from about 5% to about 10% by weight, talc in the range of from about 4% to about 24% by weight and TiO₂ in the range of from about 4% to about 24% by weight.

11. An enteric coated nutraceutical or pharmaceutical comprising, a nutraceutical or pharmaceutical active ingredient, and an enteric coating wherein the enteric coating comprises:
a food grade shellac,
an ammonium salt, and
an anionic polymer.

12. The enteric coated nutraceutical or pharmaceutical of claim 11 wherein the anionic polymer is selected from the group consisting of sodium carboxymethyl cellulose (CMC), sodium alginate and pectin.

13. The enteric coated nutraceutical or pharmaceutical of claim 12 wherein the anionic polymer comprises sodium carboxymethyl cellulose (CMC) in an amount in the range of from about 1% to about 18% by weight of the enteric coating.

14. The enteric coated nutraceutical or pharmaceutical of claim 11 wherein the food grade shellac is Orange Dewaxed Shellac in an amount in the range of from about 20% to about 75% by weight of the enteric coating.

15. The enteric coated nutraceutical or pharmaceutical of claim 11 wherein the enteric coating further comprises one or more plasticizers chosen from the group consisting of glycerine, mineral oil, triacetin polyethylene glycol, glyceryl monostearate, mono acetylated triglyceride and polysorbate.

16. The enteric coated nutraceutical or pharmaceutical of claim 11 wherein the enteric coating further comprises an inorganic pigment in an amount up to about 70% by weight of the weight of the enteric coating.

17. A process for producing a sprayable dispersion enteric coating comprising the steps of:
dry blending a food grade shellac, an ammonium salt, an anionic polymer, one or more plasticizers chosen from glycerine, mineral oil, triacetin, polyethylene glycol, glyceryl monostearate, mono acetylated triglyceride and polysorbate, and, optionally, pigments, and detackifiers such as titanium dioxide, talc, iron oxides and natural colors together to form a dry powder formulation,
dispersing the dry powder formulation in about 50 to 70° C. hot water, and stirring the dispersed the dry powder formulation for a sufficient period of time to produce a low viscosity sprayable dispersion wherein the low viscosity sprayable dispersion at 15% solids in water has a viscosity of below 100 cps at about 22° C. when measured with a Brookfield LVT viscometer with a #1 spindle at 100 rpm.

18. A process for producing a solid dosage form having an enteric coating comprising the steps of:
obtaining a nutraceutical or pharmaceutical active ingredient in a solid dosage form,
dry blending a food grade shellac, an ammonium salt, an anionic polymer, one or more plasticizers chosen from glycerine, mineral oil, triacetin, polyethylene glycol, glyceryl monostearate, mono acetylated triglyceride and polysorbate, and, optionally, and, optionally, pigments, and detackifiers such as titanium dioxide, talc, iron oxides and natural colors together to form a dry powder formulation,
dispersing the dry powder formulation in about 50 to 70° C. hot water,
stirring the dispersed the dry powder formulation for a sufficient period of time to produce a low viscosity sprayable dispersion wherein the low viscosity sprayable dispersion at 15% solids in water has a viscosity of below 100 cps at about 22° C. when measured with a Brookfield LTV viscometer with a #1 spindle at 100 rpm, and
spraying the low viscosity sprayable dispersion onto the nutraceutical or pharmaceutical active ingredient in a solid dosage form to produce an enteric coating on the nutraceutical or pharmaceutical active ingredient in a solid dosage form.
